(19) United States
(12) Patent Application Publication
(10) Pub. No.: US 2026/0209676 A1
(43) Pub. Date: Jul. 23, 2026

(54) Plasma Treatment System for Microbial Activation

(71) Applicant: Meta Organic Technology Inc, Sejong-si (KR)

(72) Inventors: Yi Young Ahn, Gunsan-si (KR); Kyung Hee Han, Seoul (KR); Dong Gi Youn, Seoul (KR)

(21) Appl. No.: 19/399,716

(22) Filed: Nov. 25, 2025

(30) Foreign Application Priority Data
Jan. 22, 2025 (KR) 10-2025-0009315
Sep. 29, 2025 (KR) 10-2025-0141035

(51) Int. Cl. C12M 1/42; C12M 1/00; C12M 1/06; C12M 1/34

(52) U.S. Cl. CPC C12M 35/08; C12M 27/06; C12M 29/08; C12M 41/26; C12M 41/34; C12M 41/38

(57) ABSTRACT

A plasma treatment system for microorganisms includes a plasma generation unit that generates reactive gas through plasma discharge; a microbial treatment unit including a chamber having an internal space for accommodating a culture medium containing the microorganisms, an inlet into which the reactive gas from the plasma generation unit is injected, and an outlet for discharging the gas generated in the internal space, in which the microbial treatment unit includes: a bubbler that disperses the reactive gas supplied through the inlet in a form of bubbles; and a blade that agitates the culture medium within the chamber, and the bubbler and the blade are provided adjacent to a bottom surface of the chamber.

### CROSS-REFERENCE TO RELATED APPLICATIONS

This application claims priority to Korean Patent Application No. 10-2025-0009315 filed January 22, 2025, and to Korean Patent Application No. 10-2025-0141035 filed September 29, 2025, the disclosures of which are hereby incorporated herein by reference in their entireties.

### STATEMENT REGARDING SPONSORED RESEARCH OR DEVELOPMENT

This invention was made with support by the Starting growth Technological R&D Program (TIPS Program, (No. RS-2025-00282748) funded by the Ministry of SMEs and Startups(MSS, Korea) in 2025.

Unique Project ID (Assigned by NTIS): 2420023818

Project Number (Sub-project No.): RS-2025-00282748

Ministry: Ministry of SMEs and Startups (MSS), Republic of Korea

Research Management Agency: Korea Technology and Information Promotion Agency for SMEs (TIPA)

R&D Program: Startup Growth Technology Development Program (TIPS Program)

Project Title: Premium agricultural growth element prescription and management platform based on real-time soil AIoT data

Host Institution: meta organic technology INC

Project Period: July 1, 2023 - June 30, 2026

### BACKGROUND

### TECHNICAL FIELD

The following disclosure relates to a plasma treatment system for microorganisms and a method for microbial activation using plasma treatment.

### TECHNICAL CONSIDERATIONS

Plasma is the fourth state of matter, and is a high- energy state after solid, liquid, and gas. Specifically, the plasma state is a state in which, when high energy is applied to a gas, the molecules become ionized, resulting in a mixture of electrons, ions, and neutral particles. The plasma is electrically neutral, and characterized by high energy and reactivity. Technologies make use of the characteristics of plasma by applying high voltage or high-frequency energy to a gas to generate the plasma, which is then employed to treat various matters. The plasma contains activated chemical and physical elements such as reactive oxygen species (ROS), reactive nitrogen species (RNS), ions, radicals, and ultraviolet (UV) light. These elements may be utilized to modify surfaces of matters or induce chemical reactions.

Conventional plasma technology has been utilized in various industrial and environmental fields for a long period of time. Representatively, the plasma technology has played a key role in chemical synthesis processes such as nitrogen fixation and ammonia synthesis. These technologies are based on the property of plasma to promote reactions between gas molecules or chemicals at high temperatures and high energy levels. In addition, the plasma exhibits outstanding performance in sterilization and purification technologies, and contributes to resolving environmental and sanitary issues such as pathogen removal and decomposition of hazardous substances.

However, the applications of the existing plasma technology are primarily limited to chemical reactions and physical treatments, and the application to biological systems is still in its early stages. In particular, technologies utilizing plasma reactive species to control or promote microbial metabolic reactions have not yet been clearly defined. This is due to the complex impact of plasma-generated reactive oxygen species (ROS) and reactive nitrogen species (RNS) on microbial physiological and biochemical mechanisms, and limited understanding of their interactions.

Therefore, there is a growing need for the development of technologies that can apply plasma reactive species to biological systems.

### SUMMARY

A non-limiting embodiment of the present disclosure is directed to providing a system for activating microbial metabolism capable of controlling or promoting microbial metabolic reaction using plasma reactive species.

A non-limiting embodiment of the present disclosure is directed to providing a method for activating microbial metabolism capable of enhancing microbial metabolic reaction using plasma reactive species

Aspects of the present disclosure are not limited to the above-described aspects. That is, other aspects that are not described may be obviously understood by those skilled in the art from the following specification.

In one general non-limiting aspect, a plasma treatment system for microorganisms includes: a plasma generation unit that generates reactive gas through plasma discharge; a microbial treatment unit including a chamber having an internal space for accommodating a culture medium containing the microorganisms, an inlet into which the reactive gas from the plasma generation unit is injected, and an outlet for discharging the gas generated in the internal space, in which the microbial treatment unit includes: a bubbler that disperses the reactive gas supplied through the inlet in a form of bubbles; and a blade that agitates the culture medium within the chamber, and the bubbler and the blade are provided adjacent to a bottom surface of the chamber.

The microbial treatment unit may further include a collision plate that is provided above the blade and has an open area at one side, the bubbler, the blade, and the collision plate may be sequentially stacked within the chamber from the bottom surface of the chamber.

The collision plate may be provided in plurality, and may be provided to form a curved movement path for the culture medium.

The collision plate may include: a first collision plate contacting a first surface of one of facing inner walls of the chamber and spaced apart from a second surface; and a second collision plate contacting the second surface and spaced apart from the first surface, and the first collision plate and the second collision plate may be alternately provided along a vertical direction of the chamber.

The collision plate may have a streamlined curved cross-section and may be provided to have a predetermined upward inclination angle relative to a horizontal plane.

A surface of the collision plate may be coated with a photocatalytic material.

The microbial treatment unit may further include an ultraviolet irradiation unit that irradiates ultraviolet light to activate the photocatalytic material, and the ultraviolet irradiation unit may be provided close to the collision plate.

The ultraviolet irradiation unit may be provided on the facing inner walls of the chamber.

The microbial treatment unit may further include a heater that controls a temperature of the culture medium within the chamber, and the heater may be provided on the bottom surface of the chamber.

The microbial treatment unit may further include: a sensor that measures at least one of dissolved oxygen content, pH, or specific metabolite concentration of the culture medium; and a control unit that controls the plasma generation unit, and the control unit may be provided to adjust an activated species generation condition of the plasma generation unit based on a value measured from the sensor.

### BRIEF DESCRIPTION OF THE DRAWINGS

FIG. 1 is a diagram illustrating a plasma treatment system for microorganisms according to a non-limiting embodiment of the present disclosure;

FIG. 2 is a cross-sectional view taken along line A-A' of FIG. 1; and

FIG. 3 is a flowchart of a method for microbial metabolism activation according to one non-limiting embodiment of the present disclosure.

### DETAILED DESCRIPTION

The present disclosure is described in detail below. Unless otherwise defined, the terms used herein shall be construed as having the meanings generally understood by those of ordinary skill in the art. The drawings and non-limiting embodiments of this specification are provided to enable those skilled in the art to easily understand and practice the present disclosure. Contents that may obscure the gist of the disclosure may be omitted from the drawings and examples, and the present disclosure is not limited to the drawings and non-limiting embodiments.

The singular forms used in the specification are intended to include the plural forms as well, unless the context specifically dictates otherwise.

In this specification, the terms "include", "have", "comprise", etc., means that features or components described in the specification are present, and unless specifically limited, it does not preclude in advance the possibility that one or more other features or components may be added.

A plasma treatment system 1000 for microorganisms of the present disclosure provides a plasma treatment system 1000 for microorganisms that may control a concentration of reactive species and enhance a metabolic response of microorganisms through plasma treatment.

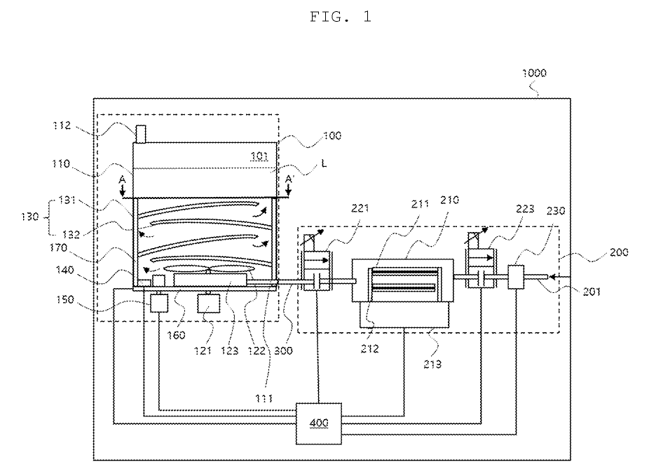

FIG. 1 is a diagram illustrating the plasma treatment system 1000 for microorganisms according to a non-limiting embodiment of the present disclosure. Referring to FIG. 1, the plasma treatment system 1000 for microorganisms of the present disclosure includes an injection unit 300, a control unit 400, a power supply device (not illustrated), a microbial treatment unit 100, and a plasma generation unit 200.

The injection unit 300 supplies reactive species from the plasma generation unit 200 to the microbial treatment unit 100. For example, the injection unit 300 may be manufactured as a tube with excellent heat resistance and chemical stability, and is provided to connect an outlet of a plasma discharge tube 210 and an inlet 111 of the chamber 110.

The control unit 400 controls a general operation of the system 1000. In one example, the control unit 400 receives data such as temperature, pressure, pH, and dissolved oxygen from a sensor 140 in real time, and compares the data with set valves to comprehensively control operations of a high-pressure generator 213, a plurality of valves 221 and 223, a pump 230, a motor 121, a heater 160, etc., thereby automatically maintaining optimal conditions for microbial culture and plasma treatment. In one example, the control unit 400 may include a microcontroller.

The power supply device (not illustrated) stably supplies operating power required for each component of the system 1000 (control unit 400, high-pressure generator 213, motor 121, pump 230, heater 160, etc.).

The microbial treatment unit 100 includes, for example, a chamber 110, an inlet 111, an outlet 112, a bubbler 123, a blade 122, a motor 121, a collision plate 130, a sensor 140, an oxygen generator 150, a heater 160, and an ultraviolet irradiation unit 170.

The chamber 110 is provided to accommodate a culture medium containing microorganisms in an internal space 101. In one example, the chamber 110 is provided with a sealable structure. In one example, the chamber 110 is manufactured from a material having corrosion resistance and heat resistance so as to be stable against chemical and physical changes during the plasma reactive species and the culture process. In one example, the material of the chamber 110 may include, but is not limited to, at least one of stainless steel, borosilicate glass, quartz, and polyether ether ketone (PEEK).

The inlet 111 and the outlet 112 are provided to supply reactive gas to the chamber 110 and discharge the gas inside. In one example, the inlet 111 is connected to the injection unit 300 and receives reactive species generated from the plasma generation unit 200. In one example, the outlet 112 discharges gases or byproducts remaining after reaction with the culture medium to the outside of the chamber 110.

In one example, the inlet 111 is provided on a bottom surface of the chamber 110 or on a side surface adjacent to the bottom surface. In one example, the outlet 112 is provided above the chamber 110. In one example, the inlet 111 may be installed through the bottom surface of the chamber 110 or the side surface adjacent to the bottom surface.

The bubbler 123 primarily disperses the reactive gas supplied into the chamber 110 through the inlet 111 into the culture medium in the form of fine bubbles. In one example, the inlet 111 is connected to the bubbler 123. The gas supplied through the inlet 111 is immediately converted into fine bubbles as the gas passes through the bubbler 123, allowing the gas to be effectively dispersed into the culture medium. In one example, the bubbler 123 is arranged on the bottom surface of the chamber 110. By positioning the bubbler 123 on a lower side of the chamber 110 and connecting the inlet 111 to the bubbler 123, the reactive gas has the longest possible movement path through the culture medium, thereby maximizing the residence time of the reactive gas within the culture medium.

The blade 122 is a plurality of blades connected to a rotational axis of the motor 121 and rotating together. In one example, the blade 122 is arranged on the lower side of the chamber 110, similar to the bubbler 123. In one example, the blade 122 is arranged above the bubbler 123, physically breaking up the gas bubbles dispersed through the bubbler 123 into finer pieces and forcibly mixing the gas bubbles with the culture medium. This mechanically enhances the dissolution efficiency of the reactive gas into the culture medium.

By organically arranging the inlet 111, bubbler 123, and blade 122 on the lower side of the chamber 110, the delivery efficiency of the reactive species is enhanced. Specifically, the gas inlet 111 is arranged on the lower side, the lowest point of the chamber 110, so that the injected reactive gas travels through the entire height of the culture medium to reach the upper outlet 112. This physically maximizes the residence time and movement path of gas bubbles within the culture medium, minimizing material loss due to gas being released without reacting and ensuring sufficient time for the reactive species to dissolve within the culture medium. Furthermore, the step-wise interaction of the components arranged on the lower side increases the amount of gas dissolved per unit time.

First, the reactive gas supplied through the inlet 111 passes through the bubbler 123 and is initially converted into finely divided bubbles, thereby increasing the initial gas-liquid contact area. Subsequently, the blade 122 rotating at high speed above the bubbler 123 physically breaks these bubbles into even finer microbubbles through strong shear force, while simultaneously forming a strong vortex at the bottom of the chamber 110 to forcibly convect the microbubbles throughout the culture medium. This mechanical agitation resolves the concentration gradient within the culture medium, ensuring that reactive species, temperature, nutrients, etc., are uniformly distributed throughout the chamber 110. This prevents a phenomenon in which reactive species are concentrated only in specific regions, causing microorganisms to experience localized oxidative stress, and ensures that all microorganisms are placed under consistent treatment conditions.

Consequently, this arrangement organically combines the technical benefits of securing the longest reaction time, maximizing the contact area through multi-step physical action, and ensuring a uniform culture environment. This increases the total amount of injected reactive species delivered to the microorganisms, ultimately enhancing the microbial metabolic reaction.

The motor 121 is installed at a lower end of the chamber 110 and generates power to rotate the blade 122. In one example, the motor 121 may be a high-speed motor capable of high-speed rotation, and its rotation speed may be adjusted according to a control signal from the control unit 400.

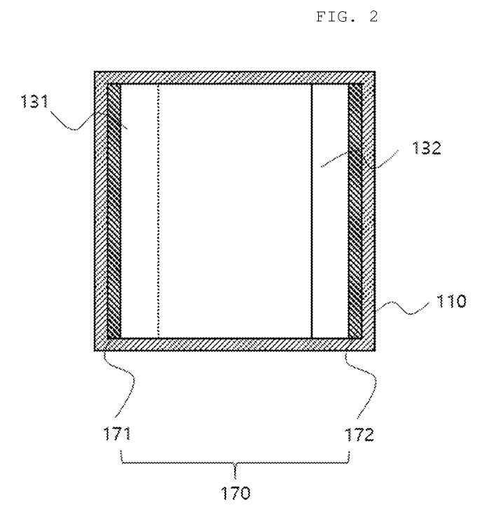

FIG. 2 is a cross-sectional view taken along line A-A' of FIG. 1. Referring to FIG. 2, the collision plate 130 may be seen when viewing the chamber 110 from above.

In one example, a plurality of collision plates 130 are provided and arranged in multiple layers within the chamber 110. The collision plate 130 is provided above the blade 122. For example, the plurality of collision plates 130 are arranged in an alternating manner on the rising path of the gas. In one example, the plurality of collision plates 130 include a first collision plate 131 and a second collision plate 132. The first collision plate 131 is fixed in contact with a first surface of the facing inner walls of the chamber 110 and is spaced apart from the second surface by a predetermined distance. Conversely, the second collision plate 132 is fixed in contact with the second surface and is spaced apart from the first surface by a predetermined distance. In one example, the first surface and the second surface are connected through the bottom surface of the chamber 100 to form a side wall of the chamber 100.

Alternatively, the ultraviolet irradiation unit 170 is provided on a first surface and a second surface of the chamber 110, respectively, and the first collision plate 131 is fixed in contact with the first ultraviolet irradiation unit 170, and the second collision plate 132 is fixed in contact with the second ultraviolet irradiation unit 170. It is preferable that the ultraviolet irradiation unit 170 is arranged close to the collision plate 130 coated with a photocatalyst in order to minimize the light loss caused by ultraviolet rays being absorbed or scattered by the culture medium and to maximize the density of photons reaching the surface of the photocatalyst. The ultraviolet irradiation unit 170 may be provided as a panel in the form of a surface light source attached to the inner wall of the chamber 110, and may have a 'light source-catalyst integrated' structure in which the collision plate 130 coated with the photocatalyst is fixed in direct contact with the panel. Alternatively, the UV irradiation unit 170 may be a thin, rod-shaped UV lamp or a linear UV-LED module, and may be alternately arranged between the collision plates 130.

By positioning the ultraviolet irradiation unit 170 in close proximity to the collision plate 130, the distance between the light source and the catalyst surface is minimized, thereby inducing an efficient photocatalytic reaction with lower energy compared to external irradiation methods and maximizing the generation of secondary reactive species such as hydroxyl radicals.

The first collision plate 131 and the second collision plate 132 are alternately arranged along the vertical direction (vertical direction) of the chamber 110. Due to this arrangement structure, gas bubbles rising from the bottom of the chamber 110 may not rise in a straight line, but should first pass through the space between the first collision plate 131 and the second surface, and then again through the space between the second collision plate 132 and the first surface, located above. As this process is repeated, the overall ascending path of the gas takes on a zigzag or serpentine shape.

As a result, the gas bubbles travel along a longer path, rather than the shortest straight line from the lower chamber 110 to the upper outlet 112, thereby increasing their residence time within the culture medium. Furthermore, the repeated passage through the narrow passage and the continuous change in direction of movement induce turbulence, further enhancing the contact efficiency and mass transfer efficiency between the reactive gas and the culture medium.

In one example, the collision plate 130 may have a streamlined, convex cross-section. This induces rising gas bubbles to flow smoothly along the surface of the collision plate 130, thereby suppressing the occurrence of dead zones where gas stagnates and allowing the entire surface of the collision plate 130 to effectively participate in the reaction.

In one example, each collision plate 130 may be arranged to have a predetermined upward inclination angle relative to the horizontal plane. This inclination angle serves to actively induce the gas bubbles not only horizontally but also toward the center of the chamber 110 or a specific location on the next collision plate 130.

In one example, each collision plate 130 may be coated with a photocatalytic material on at least one of the surface or the bottom surface and the top surface, which are in active contact with the gas. In one example, the photocatalytic material may include titanium dioxide (TiO₂ ).

Referring back to FIG. 1, the sensor 140 is provided to measure environmental variables within the chamber 110 in real time. In one example, the sensor 140 is mounted on a sensor 140 port within the chamber 110 to measure the temperature, pressure, pH, dissolved oxygen (DO), etc. of the culture medium and to transmit the measurement data to the control unit 400.

An oxygen generator 150 may be provided to supply oxygen necessary for metabolic reaction when the cultured microorganism is an aerobic microorganism. Separately from the supply of plasma reactive species, the oxygen generator 150 may serve to maintain the dissolved oxygen level within the culture medium at an optimal level under the control of the control unit 400.

The heater 160 is provided to maintain the temperature of the culture medium within the chamber 110 at an optimal growth temperature for the microorganism. In one example, the heater 160 is arranged adjacent to the bottom surface of the chamber 110. In one example, the heater 160 may be mounted on the bottom surface of the chamber 110. When the heater 160 is arranged on the bottom surface, it may synergize with the stirring action of the blade 122 below to rapidly circulate the heated culture medium throughout the chamber 110. Alternatively, the heater 160 may be provided in the form of a jacket that wraps around the outer wall of the chamber 110. This allows for even heat transfer to the entire culture medium and prevents local overheating that may cause thermal damage to microorganisms. The operation of the heater 160 is controlled by the control unit 400 based on the temperature value measured by the sensor 140.

The plasma generation unit 200 includes a gas supply unit 201, a plasma discharge tube 210, a high-pressure generator 150, a plurality of valves 221 and 223, a pump 230, and a high- pressure generator 213.

The gas supply unit 201 stores raw gas (e.g., oxygen, nitrogen, argon, helium, etc.) to be used for plasma discharge and supplies the raw gas to the plasma discharge tube 210.

The plasma discharge tube 210 generates reactive species selected from active oxygen species and active nitrogen species through plasma discharge. In one example, the plasma discharge tube 210 may stably generate plasma in an atmospheric pressure environment using dielectric 211 discharge technology (dielectric barrier discharge (DBD)). Specifically, the plasma discharge tube 210 may be composed of two facing electrodes 212 and a dielectric 211 therebetween, and forms plasma by applying a high voltage in the form of an alternating current or pulse to the electrodes 212. In this case, the reactive species generated may be one or more selected from the group consisting of nitrogen monoxide, nitrogen dioxide, nitrate, nitrite, nitrogen radicals, ozone, hydroxyl radicals, and hydrogen peroxide.

The high-voltage generator 213 supplies high-voltage power to the electrode 212 of the plasma discharge tube 210. The high-voltage generator 213 receives a control signal from the control unit 400 and adjusts the intensity of the output voltage, thereby actively controlling the generation intensity of the plasma, i.e., the concentration of the generated reactive species.

The plurality of valves 221 and 223 are provided to precisely control the flow of gas. In one example, the valves 221 and 223 include a first valve 221 and a second valve 223. The first valve 221 is a proportional control solenoid valve installed in the injection unit 300, and precisely controls the flow rate, speed, and pressure of the reactive gas supplied to the microbial treatment unit 100 by finely adjusting the degree of opening of the valves 221 and 223 according to a signal from the control unit 400. The second valve 223 is a proportional control solenoid valve located in a line provided with a gas supply unit 201 or an outlet 112, and may serve to control the flow of gas.

The pump 230 supplies gas from the gas supply unit 201 to the plasma discharge tube 210 at a constant pressure. The pump 230 may be used to comprehensively control the concentration of reactive species, along with the voltage regulation of the high- pressure generator 213, through the control unit 400.

In one example, the plasma treatment system 1000 for microorganisms of the present disclosure may have a modular structure.

This means that the main components of the system, namely the microbial treatment unit 100, the plasma generation unit 200, and the control unit 400, are each composed of independent physical units. Each unit may be detachably connected to another unit through distinct mechanical and electrical interfaces.

Therefore, the user may configure a customized system by selectively combining each unit depending on the type of microorganism to be treated or the purpose of the treatment. Furthermore, when a specific unit malfunctions or performance improvement is required, only the unit may be easily replaced or upgraded without disassembling the entire system, significantly increasing the operational flexibility and expandability of the system.

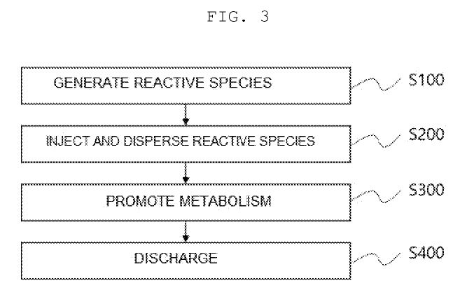

Next, a method for microbial metabolism activation using the plasma treatment system for microorganisms described above will be described. FIG. 3 is a method for microbial metabolism activation according to one non-limiting embodiment of the present disclosure. Referring to FIG. 3, the method for microbial metabolism activation according to a non-limiting embodiment of the present disclosure may include a reactive species generation step (S100), a reactive species injection and dispersion step (S200), a metabolism promotion step (S300), and a discharge step (S400). The control unit 400 is provided to perform each step.

In the reactive species generation step (S100), the plasma generation unit 200 generates the reactive species according to a control signal from the control unit 400. Specifically, the control unit 400 opens the valves 221 and 223 of the gas supply unit 201 to supply a raw material gas selected from oxygen, nitrogen, argon, helium, etc., to the plasma discharge tube 210 through the pump 230. Simultaneously, the high-pressure generator 213 applies a high voltage to the electrode 212 of the plasma discharge tube 210 to generate plasma, thereby generating the reactive gas including active oxygen species and active nitrogen species. In this case, the control unit 400 may control the concentration of the generated reactive species by adjusting the output voltage of the high-pressure generator 213 and the operation of the pump 230.

In the reactive species injection and dispersion step (S200), the generated reactive gas is injected into the lower portion of the chamber 110 of the microbial treatment unit 100 through the injection unit 300. The injected reactive gas is initially dispersed into fine bubbles as it passes through the bubbler 123 located on the bottom surface of the chamber 110. Subsequently, the gas is physically further broken down by a high- speed rotating blade 122 above the bubbler 123 and forcibly stirred with the culture medium to ensure uniform dispersion within the chamber 110. In this step, the control unit 400 precisely controls the rotation speed of the motor 121 and the proportional control solenoid valves 221 and 223 of the injection unit 300 to precisely adjust the flow rate, speed, and degree of dispersion of the reactive species injected into the culture medium.

In the metabolic promotion step (S300), the reactive species evenly dispersed within the culture medium contacts microorganisms, inducing various biochemical reactions such as altering the permeability of cell membranes or regulating the activity of specific enzymes, thereby promoting microbial growth and metabolic reaction.

In particular, this step may be performed by allowing gas bubbles passing through the blade 122 to ascend along a complex, zigzag path formed by the multi-layer collision plate 130. This maximizes the residence time within the culture medium of gas, thereby enhancing the reaction efficiency between the reactive species and microorganisms.

If the surface of the collision plate 130 is coated with a photocatalyst, the photocatalyst may be activated by the ultraviolet irradiation unit 170, additionally generating reactive species having high reactivity, such as hydroxyl radicals, within the chamber 110. In this case, the combined action of externally injected reactive species and internally generated reactive species may further amplify the metabolic promotion.

Throughout this entire process, the control unit 400 monitors environmental data, such as temperature, pH, and dissolved oxygen content, in real time from sensors 140 within the chamber 110 and controls the operation of the heater 160 and oxygen generator 150, thereby maintaining an optimal environment for microbial metabolic reaction.

Finally, in the discharge step (S400), any remaining gas after the reaction with the microorganisms and any gaseous byproducts generated during the metabolic process are safely discharged to the outside of the system through the outlet 112 located at the top of the chamber 110.

The present disclosure organically combines the multi- stage physical actions of the bubbler, blades, and the collision plate with the chemical actions of the photocatalyst and UV irradiator to maximize the delivery of reactive species and reaction efficiency, thereby effectively promoting microbial metabolic reaction.

Furthermore, the present disclosure maintains a uniform culture medium environment through mechanical agitation and actively controls process variables through real-time sensor feedback, thereby preventing microbial damage and achieving stable, highly reproducible results.

In addition, according to the present disclosure, each functional unit is configured as an independent module, so that a customized system configuration suitable for the intended use is possible, and since replacement and upgrade of specific modules are easy, there is an advantage in that the operational flexibility and economy of the system may be increased.

The plasma treatment system for microorganisms according to a non-limiting embodiment of the present disclosure can efficiently implement the plasma treatment on microorganisms by minimizing losses during the reactive species delivery process, and furthermore, can be applied to the bio and environmental technology fields to enhance the microbial metabolism and produce the special metabolites.

The method for microbial activation according to a non- limiting embodiment of the present disclosure can improve the microbial growth and metabolic rates and enhance the energy efficiency.

Effects of the present disclosure are not limited to the above-described effects, and effects that are not mentioned will be clearly understood by those skilled in the art to which the present disclosure pertains from the present specification and the accompanying diagrams.

Hereinabove, although the present disclosure has been described by specific matters and limited examples and comparative examples, they have been provided only for assisting in the entire understanding of the present disclosure. Therefore, the present disclosure is not limited to the embodiments. Various modifications and changes may be made by those skilled in the art to which the present disclosure pertains from this description.

Therefore, the spirit of the present disclosure should not be limited to the above-described exemplary embodiments, and the following claims as well as all modified equally or equivalently to the claims are intended to fall within the scopes and spirits of the disclosure.

What is claimed is:

1. A plasma treatment system for microorganisms, comprising:
a plasma generation unit that generates reactive gas through plasma discharge; and
a microbial treatment unit comprising a chamber having an internal space for accommodating a culture medium containing the microorganisms, an inlet into which the reactive gas from the plasma generation unit is injected, and an outlet for discharging the gas generated in the internal space,
wherein the microbial treatment unit comprises:
a bubbler that disperses the reactive gas supplied through the inlet in a form of bubbles; and
a blade that agitates the culture medium within the chamber, and
the bubbler and the blade are provided adjacent to a bottom surface of the chamber.

2. The plasma treatment system of claim 1, wherein the microbial treatment unit further comprises a collision plate that is provided above the blade and has an open area at one side, and
the bubbler, the blade, and the collision plate are sequentially stacked within the chamber from the bottom surface of the chamber.

3. The plasma treatment system of claim 2, wherein the collision plate is provided in plurality, and is provided to form a curved movement path for the culture medium.

4. The plasma treatment system of claim 2, wherein the collision plates comprise:
a first collision plate contacting a first surface of one of facing inner walls of the chamber and spaced apart from a second surface; and
a second collision plate contacting the second surface and spaced apart from the first surface, and
the first collision plate and the second collision plate are alternately provided along a vertical direction of the chamber.

5. The plasma treatment system of claim 2, wherein the collision plate has a streamlined curved cross-section and is provided to have a predetermined upward inclination angle relative to a horizontal plane.

6. The plasma treatment system of claim 2, wherein a surface of the collision plate is coated with a photocatalytic material.

7. The plasma treatment system of claim 6, wherein the microbial treatment unit further comprises an ultraviolet irradiation unit that irradiates ultraviolet light to activate the photocatalytic material, and
the ultraviolet irradiation unit is provided close to the collision plate.

8. The plasma treatment system of claim 7, wherein the ultraviolet irradiation unit is provided on the facing inner walls of the chamber.

9. The plasma treatment system of claim 1, wherein the microbial treatment unit further comprises a heater that controls a temperature of the culture medium within the chamber, and
the heater is provided on the bottom surface of the chamber.

10. The plasma treatment system of claim 1, wherein the microbial treatment unit further comprises:
a sensor that measures at least one of the following: dissolved oxygen content, pH, specific metabolite concentration of the culture medium, or any combination thereof; and
a control unit that controls the plasma generation unit, and
the control unit is provided to adjust an reactive species generation condition of the plasma generation unit based on a value measured from the sensor.
